(19) United States
(12) Patent Application Publication
(10) Pub. No.: US 2015/0344847 A1
(43) Pub. Date: Dec. 3, 2015

(54) Method For Production Of Large Numbers Of Cartilage Cells With Phenotype Retention

(71) Applicant: Theodore I Malinin, Key Biscayne, FL (US)

(72) Inventor: Theodore I Malinin, Key Biscayne, FL (US)

(73) Assignee: VIVEX BIOMEDICAL INC., Marietta, GA (US)

(21) Appl. No.: 14/290,438

(22) Filed: May 29, 2014

(51) Int. Cl. C12N 5/077

(52) U.S. Cl. CPC C12N 5/0655; C12N 2533/90; C12N 2501/734; C12N 2533/70; C12N 2533/78; C12N 2533/30; C12N 2533/54

(57) ABSTRACT

A method for production of large numbers of cartilage cells with phenotype retention intended for treatment of articular cartilage lesions or preparing viable cartilage tissue by propagation of chondrocytes from cartilage explants, human or animal, with the retention of phenotypes of the cartilage cells, in which cells are chondrocytes retaining morphologic attributes of the same or chondrocyte progenitor cells. The method includes culturing the cells in which cultured cells are organized into mature hyaline cartilage on the surface of biologic structures such as cancellous or cortical bone lamina. The method for culturing chondrocytes to produce three dimensional cellular structures consisting of cartilage cells and cell produced extracellular cartilage matrix.

### TECHNICAL FIELD

The present invention relates to the propagation of cartilage cells with phenotype retention in large quantities. Unlike many conventional cell and tissue culture techniques the method of the invention does not depend on the enzymatic, mechanical or chemical segregation of cells. Instead disclosure in the invention reveals the method of the production of cells in virtually unlimited quantities to be employed in the repair of articular cartilage defects. The cells produced are intended for use in the treatment of articular lesions. The method provides for the formation of new hyaline cartilage.

### BACKGROUND OF THE INVENTION

Numerous people suffer from various forms of arthritis including single isolated lesions which limit normal joint function and result in chronic pain and in loss of quality of life. Loss of articular cartilage results in the joint stiffening and pain due to exposure of nerve ending in the subchondral bone, and the bone on bone articulation. Unlike other tissues; adult cartilage cannot repair itself. Lack of blood supply is largely responsible for restricting cartilage ability to mobilize chondroprogenitor cells that could repair articular cartilage defects. Consequently when articular cartilage lesions progress, the patients are doomed either to chronic disability or to arthroplastics with metallic and plastic prostheses. However, the latter wear out with time, necessitating revision.

Tissue-engineered repair of articular cartilage offers a biologic alternative which may delay, reduce or eliminate the need for metal, ceramic and polymer-based materials currently employed in joint arthroplasties. Several biologic means have been investigated as means of regenerating damaged or diseased articular cartilage. With the exception of non-viable articular cartilage particles taught in U.S. Pat. No. 8,318,212 by the inventor Malinin which stimulate regeneration of the cartilage by recruitment of chondroprogenitor cells of the host, none have met with unequivocal success.

Cartilage regeneration is problematic. Unlike bone damaged cartilage does not regenerate. For these reasons, replacement of the damaged articular cartilage with artificial joints is common.

Chondrogenesis is a complex biological process having distinct beneficial medical potentials. Innumerable cartilage transplantation procedures are performed annually in the U.S. Grafting procedures performed with allografts have drawbacks when compared to the transplantation of autografts with viable cells.

Cartilage transplantation is more problematic than that of bone; although allografts with viable cartilage are clinically successful, they are in short supply. The present invention would obviate this problem by providing a virtually unlimited number of scaffoldings with cartilage cells.

One technique that showed the most promise for articular cartilage regeneration is autologous chondrocyte implantation. In this procedure, a small cartilage biopsy obtained from the patient's own joint is taken to the laboratory where cartilage cells are isolated and expanded in vitro for subsequent re-implantation into the patient. A significant limitation of this method is the relatively small number of donor cells that can be obtained by biopsy. Furthermore, chondrocytes from adult articular cartilage appear to have a limited ability to produce cartilage matrix after expansion, i.e. loss of the original chondrocyte attributes. To increase the production of cartilage, modification monolayer culture techniques for chondrocyte expansion have been developed, as taught in Kuettner U.S. Pat. No. 4,356,261. However, chondrocytes propagated in monolayer cultures using serum-containing media undergo a process of transformation dedifferentiation in which they lose their spherical shape and acquire an elongated fibroblastic morphology. This is typical of cell cultures, biochemical changes associated with loss of native chondrocyte shape include arrested synthesis of cartilage-specific collagens and proteoglycans, subsequent initiation of type I and III collagen synthesis and increased synthesis of small non-aggregating proteoglycans.

Loss of chondrocyte phenotype during serial expansion in vitro poses a definite limitation to the development of orthobiologic articular cartilage repair. In attempts to counter dedifferentiation, (transformation) chondrocytes have been cultured in three dimensional systems and are taught in various publications, such as agarose, Benya and Shafer, 1982; alginate, Hauselmann et al, 1994; pellet culture, Jacob et al 2001; or three-dimensional scaffolds, Vacanti et al., 1998. Chondrocytes were reported to better retain their native rounded morphology and to synthesize macromolecules characteristic of hyaline cartilage when maintained in three-dimensional cultures. However, such cultured chondrocytes still produced type I collagen and small proteoglycans, indicating an “incomplete” cartilage phenotype.

Numerous patents, patent application and scientific publications are devoted to chondrocyte culture in vitro and use of such cultured chondrocytes for the treatment of articular cartilage defects. However, most of these endeavors are directed towards in vitro expansion of chondrocytes. Because of the large numbers of the material, only a few examples can be cited without producing a voluminous treatise.

Adkinson et al (US20080081369/A1) provide a method for growing chondrocytes while maintaining chondrocyte phenotype. The method comprises culturing a population of chondrocytes in a serum free culture medium containing cytokines on a substratum containing cytokines, on a substratum comprising tissue culture plastic to which hyaluronic acid is covalently attached. The invention addresses loss of chondrocyte phenotype during serial expansion in vitro which poses a key limitation to the commercialization of orthobiologic approaches to articular cartilage repair. The invention has only a few peripheral relations to the present invention, as the technique described is limited to the cultivation of segregated chondrocytes expansion of which inevitably leads to cellular transformation i.e. dedifferentiation. The present invention is directed towards primary cell growth not subjected to conditions which induce dedifferentiation.

Thirion and Berenbaum (Thirion S. & Berenbaum F.; Methods in Molecular Medicine, Vol. 100: Cartilage and Osteoarthritis, Vol. 1: Cellular and Molecular Tools (Sabatini M et al eds) (Humana Press, Totowa, N.J.) describe several protocols for culturing chondrocytes of various anatomical regions and from different species designed to limit dedifferentiation. Their protocols deal exclusively with enzymatically dispersed chondrocytes.

Kandel (U.S. Pat. No. 6,464,729) describes biologic material comprising a continuous layer of cartilaginous tissue reconstituted in vitro which contains components associated with cartilage mineralization. The invention describes enzymatically segregated cells placed on Millipore filter inserts. Explant technique, the object of the present invention, was not used. Otero et al describe strategies for maintaining or restoring dedifferentiated cells by culture in gels, suspension or scaffolds. The authors note the use of human chondrocytes has been problematical, since the source of the cartilage cannot be controlled, a sufficient number of cells is not readily obtainable and the phenotypic stability and proliferating capacity in adult human chondrocytes are lost quickly in serial monolayer cultures. Alternately explants cultures, usually bovine, have been used as in vitro models to study cartilage biochemistry and metabolism. However, most experimental manipulations are done more easily on isolated chondrocytes, the model employed by these investigators (Otero M et al.; Human Chondrocyte cultures as model of cartilage-specific gene regulation; Methods Mol. Med. 2005; 107:69-95). The present invention overcomes many of the cited difficulties. It is also directed to clinical transplantation rather than laboratory studies.

Moo et al studied bovine cartilage explants with an aim of studying cartilage degradation, and noted that cultures were most stable from day 2 to day 10. The findings of the present invention indicate that not only are chondrocytes cultured under described conditions are stable for up to 3 months, but they form fully organized hyaline cartilage.

Gendler (U.S. Pat. No. 5,904,716) placed segregated cultured chondrocytes on flexible demineralized perforated bone stating that once the cells begin to grow they can be transplanted into a patient. Unlike in the present invention the matrix is limited to perforated decalcified bone. Likewise formation of intact multilayered hyaline cartilage is not described.

Little has been achieved in the past in a way of replacing cartilage with tissue engineered structure. To date, the growth of new cartilage from either transplantation of autologous or allogeneic cartilage has been only partially successful. Microscopic islands of new cartilage have recently been demonstrated histologically in vivo by implanting recombinant bone morphogenic protein, as reported by J. M. Wozney, et al., Science, 242 1528-1534, 1988). Limited success has been achieved in making neocartilage using free autogenous grafts of perichondral flaps, as described by J. Upton, (Plastic and Reconstructive Surgery, 68(2), 166-174;1981).

Cheung (Cell. Dev. Biol. 21:353, 1985) teaches a method of culturing canine chondrocytes on porous hydroxyapatite ceramic granules. The cells reportedly proliferated and secreted metachromatic extracellular matrix for up to 13 months. An agarose gel matrix has also been described as suitable for the in vitro culture of human chondrocytes (Delbruck et al., Conn. Tiss. Res. 15:155, 1986). Watt and Dudhia (Differentiation 38:140, 1988) disclose a composite gel of collagen and agarose for the culture of porcine chondrocytes.

U.S. Pat. No. 5,326,357, relates to the use of a synthetic medium for chondrocyte growth in vitro.

U.S. Pat. No. 5,041,138, relates to a method for making a cartilaginous structure by use of a biocompatible, biodegradable synthetic polymer matrix for chondrocyte growth in vitro, and can be used for replacing defective or missing cartilage.

U.S. Pat. No. 5,041,138 (Vacanti et al.,) describes seeding chondrocytes on biodegradable matrices for subsequent implantation in vivo. Although this system offers the advantage of a greater surface area and exposure to nutrients, the conditions employed for culturing the chondrocytes are routine. No efforts had been made to optimize the conditions for the chondrocytes to produce collagen and other matrix substances.

U.S. Pat. No. 5,902,741 relates to a method of the proliferation and cell maturation of chondrocytes in three dimensional cultures with TGF-beta supplementation. Accordingly chondrocytes are grown on a three-dimensional framework in the presence of TGF-beta. When grown in this system chondrocytes are said to mature and form components of adult tissue analogous to its counterparts in vivo. U.S. Pat. No. 5,962,325 is basically amplification on the above cited invention.

U.S. Pat. No. 8,263,405 (Akashi et al.,) provides a new reductive-stimuli-responsive degradable gel that allows control of decomposition of the three-dimensional base material for cell culture and production of a completely biological three-dimensional cellular structure. Cell-produced extracellular matrix allows safe recovery of the cellular structure produced. A stimuli-responsive hydrogel, characterized by being produced by crosslinking a water-soluble polymer with a compound having a disulfide bond in the molecular chain is also disclosed.

Bittencourt et al., have shown alginate to be an effective scaffold for chondrocytes' growth (Bittencourt RAC et al.,) (Acta Ortop Bras. 17 (4): 242-246, 2009)

U.S. Pat. No. 6,617,161 (Luyten) discloses serum-free medium to be used for chondrocyte cultivation. Formulations which included growth factors PDGF, EGF and PGF, to which a bone morphogenetic protein, BMP-7 and cartilage derived morphogenetic protein CDMP-1, were added, provided maintenance and re-expression of proteoglycan aggrecan and type II collagen in cultured human fetal chondrocytes. The disclosed culture conditions do not include hyaluronic acid. An important component of the extracellular matrix, hyaluronic acid (HA) plays a critical role in cartilage development and in the maintenance of tissue homeostasis. The present invention which depends on cultivation and production of primary and low passage cells, before they can undergo transformation, dedifferentiation obviates the problems encountered with serial expansion and monolayer cultivation of chondrocytes. It also avoids the problems encountered with these dimensional cultures.

To date, none of the aforementioned methods of cartilage bioengineering and replacement have found wide acceptance. As can be seen from the descriptions, the methods that use chondrocyte cell growth in vitro rely upon synthetic support media matrices that are foreign to the body, resulting in problems associated with the introduction of foreign compositions into the body.

### SUMMARY OF THE INVENTION

A method for producing large numbers of cartilage cells with phenotype retention intended for treatment of articular cartilage lesions or preparing viable cartilage tissue by propagation of chondrocytes from cartilage explants, human or animal, with the retention of phenotypes of the cartilage cells, in which cells are chondrocytes retaining morphologic attributes of the same or chondrocyte progenitor cells. The method includes culturing the cells in which cultured cells are organized into mature hyaline cartilage on the surface of biologic structures such as cancellous or cortical bone lamina. The method for culturing chondrocytes to produce three dimensional cellular structures consisting of cartilage cells and cell produces extracellular cartilage matrix.

In one method, the biologic material comprising hyaline cartilage tissue is propagated on the surface of thin cancellous bone which is undecalcified, and wherein the said cancellous bone lamina is from 0.5 to 2.0 mm in thickness. The cancellous bone lamina, 0.5 to 2.0 mm thick, is preferably decalcified to render it flexible. The cancellous bone lamina can be a plate either demineralized or undemineralized which is thicker than 2.0 mm. Newly proliferating cartilage can be transplanted into a chondral defect together with the underlying substrate without expanding the cells.

In another method, the substrate on which chondrocytes proliferate from explants is a lamina of undecalcified cortical bone 0.5 to 2.0 mm thick with multiple, geometrically placed perforations. Preferably, the lamina on which explants are placed is decalcified cortical bone plate 0.5 to 2.0 mm thick with multiple, geometrically placed perforations. Ideally, all osseous substrates on which chondrocytes have proliferated from cartilage explants are cryopreserved for further transplantation. Alternatively, the cartilage explants are cultivated on a cellular dermis, pericardium, dura mater and fascia with or without perforations. The chondrocytes proliferation on soft membranes with or without perforations can be cryopreserved for future transplantation. The cartilage explants are placed on Gelfoam®, Surgicel®, Gelatin USP, and or similar materials and these may be supplemented with thrombin or lignin. The cartilage explants can be grown on membranes of collodion, polyvinylpyrrolidone, hydroxyethyl starch etc. The cartilage cells together with or without cartilage explants can be harvested mechanically, enzymatically or physically from the substrates on which they are grown, frozen and either freeze-dried or dehydrated under hypothermia and micronized for implantation into articular defects. Specifically cells and explants can be frozen with substrates and then later scraped with a blade for further processing.

In one aspect the present invention provides the method for producing in culture systems of a large number of chondrocytes with retention of phenotype. The invention relies on placing multiple small fragments of native cartilage, human or animal into culture flasks, roller tubes and/or various bioreactor and have chondrocytes migrate and proliferate from these. The culture media employed (CMRL-1415, CMRL-1460, EMEM, NCTC log etc.) is supplemented with fetal bovine serum, or other blood serums. “Chondrogenic” media, available commercially maybe also employed, see FIGS. 1 and 2.

The cells proliferate from the explants and are harvested before they undergo doubling as in a traditional cell culture definition. Cells are harvested either with or without explants from which they originated. These chondrocyte preparations can be transplanted fresh or after cryopreservation. Cells with or without explants can be also freeze dried or subjected to hypothermic dehydration. These preparations can be also used to produce micronized product for transplantation into chondral defects with an aim of stimulating chondroprogenitor cells of the host to form new cartilage. In various configurations chondrocyte explants can be selected from cartilaginous tissues from either adults or juveniles, human or animal. Specifically cartilage can be obtained from articular surfaces or from ribs.

The present invention is directed to methods of generating chondral cells that can be used in cartilage repair. The method provides for cultivation of chondrocyte population without loss of its phenotype.

Another aspect of the present invention is a method for production of a large volume of cartilage cells which retain chondrocyte phenotype during their growth. These cells can be suitable for tissue transplantation to repair articular cartilage defects.

The present invention also provides a method for shipping, thawing and injecting cartilage cell preparations into articular cartilage defects. Another aspect of the present invention provided method for cartilage explants in cell culture systems to attach to the culture vessel surface and having cells proliferate from these explants.

The cells under these conditions retain morphologic characteristics of chondrocytes. The cells together with the explants or without explants are harvested form the cell culture vessels and can be transplanted immediately. Harvested cells can be preserved for future use.

The present invention is also directed to methods of producing chondrocytes for cartilage repair without having to subject these cells to the cell expansion techniques with all of their undesirable effects. The cells under conditions revealed in the invention do not require coating of the tissue culture plastic. However, other substances such as collagen can be used to provide substrates enriched with hyaluronic acid. In these primary cultures chondrocytes proliferate while maintaining their original morphology on conventional plastic cell culture surfaces, on glass or other surfaces.

Various aspects of the present invention include methods of growing chondrocytes in primary culture while maintaining their native phenotype. In some aspects, these methods include growing chondrocytes on a substrate that comprises collagen or a substratum comprising tissue culture plastic and perforated cellophane, decalcified bone membrane, glass coverslips or equivalents thereto. In various aspects, the substrate allows for maintenance of chondrocyte morphology. Cartilaginous cell sheets produced therefrom can be safe to use in medical application and be biocompatible.

Another aspect of the present invention is the methods for generating proliferating cartilage cells suitable for transplantation into articular cartilage defects.

### DEFINITIONS

As used herein and in the claims:

The term “chondrocytes” refers to specific cells that give rise to normal cartilage tissue in vivo. These cells synthesize and deposit the supportive matrix (composed principally of collagens and proteoglycan) of cartilage.

The term “phenotype” refers to observable constant characteristics morphologic, biochemical or molecular—of a cell or tissue.

The term “cytokine” refers to an array of relatively low molecular weight, pharmacologically active proteins that are secreted by one cell for the purpose of altering either its own function(s) (autocrine effect) or those of adjacent cells (paracrine effect). Individual cytokines can have multiple biological activities.

The term “FGF” indicates the fibroblast growth factors family or related proteins, which currently numbers 22 members (in humans, FGF-1-14 and FGF—16-23). “FGF-2” refers to the basic form of fibroblast growth factor (FGF).

The term “FGF-like activity” refers to an activity of a molecule, such as a polypeptide, that acts on at least one cell type in a similar manner as the FGF molecule.

The term “TGF-3” indicates the transforming growth factor family of related proteins.

The term “explant culture” is a technique used for the isolation of cells from a piece or pieces of tissue.

Tissue harvested in this manner is called an explant. It can be a portion of any part of the tissue from an animal. In brief, the tissue is harvested in an aseptic manner, often minced, and pieces placed in a cell culture vessel containing cell culture grow media. Over time, progenitor cells migrate out of the tissue onto the surface of the culture vessel. These primary cells can be further expanded and transferred into another culture vessel.

Explant culture can also refer to the culturing of the tissue pieces themselves where cells are left in their surrounding extracellular matrix to more accurately mimic the in vivo environment.

In other aspects, the invention includes novel methods for obtaining and propagating cartilage explants in vitro on several substrates. These can be obtained from adult donors, pre-adolescent donors, and neonatal and infant donors.

### BRIEF DESCRIPTION OF THE DRAWINGS

The invention will be described by way of example and with reference to the accompanying drawings in which:

FIG. 1 shows cartilage explants placed on a thin cancellous bone plate. Proliferating cartilage cells have covered the upper portion of the substrate.

FIG. 2 shows newly formed cartilage on a thin cancellous bone plate.

FIG. 3 shows chondrocytes growing from a cartilage explant. Phase-contrast, ×30.

FIG. 4 shows chondrocytes that grow from a cartilage maintain the shape and characteristic of unaltered chondrocytes. Red color indicates cytoplasmic RNA. UV light, acridine orange ×100.

FIG. 5 shows thin bone plate covered with newly formed cartilage layer. Perforations in the lower half of the bone plate are completely overgrown with newly formed cartilage. Perforations on top of the plate are only partially covered and are still visible.

FIG. 6 shows newly formed cartilage in the trabecular spaces of cancellous bone plate.

FIG. 7 shows cancellous bone plate with prominent nests of newly formed cartilage on the surface. The bone plate is covered with a cartilage layer in between cartilage nests.

FIG. 8 shows a section through a nest of newly formed hyaline cartilage on cancellous bone. Saffron-yellow coloration indicates presence and normal proteoglycans, an attribute of hyaline cartilage. Safranin 0 and light green.

### DETAILED DESCRIPTION OF THE INVENTION

Human articular chondrocytes dedifferentiate during serial monolayer culture propagation. Once the process of dedifferentiation takes place, usually around 21 days of culturing, the cells assume fibroblast-like appearance and produce predominately type 1 collagen. The mechanism of such dedifferentiation is still unknown according to Markowitz et al, FASEB Journal 14″A34; 2001.

Many attempts have been made to induce cells to re-express their differentiated phenotype in various culture systems including alginate beads, hydrogel, collagen etc. Stimulation was provided by various growth factors such as growth hormone, cytokines, TGF-beta, chondrocyte and platelet derived growth factors and several others. However, no clear cut results which might lead to therapeutic applications of the cells have been achieved thus far. Dedifferentiated chondrocytes show similar gene expression to fibroblast in monolayer cultures and the biological effect of these cells, even the autologous ones, is still uncertain.

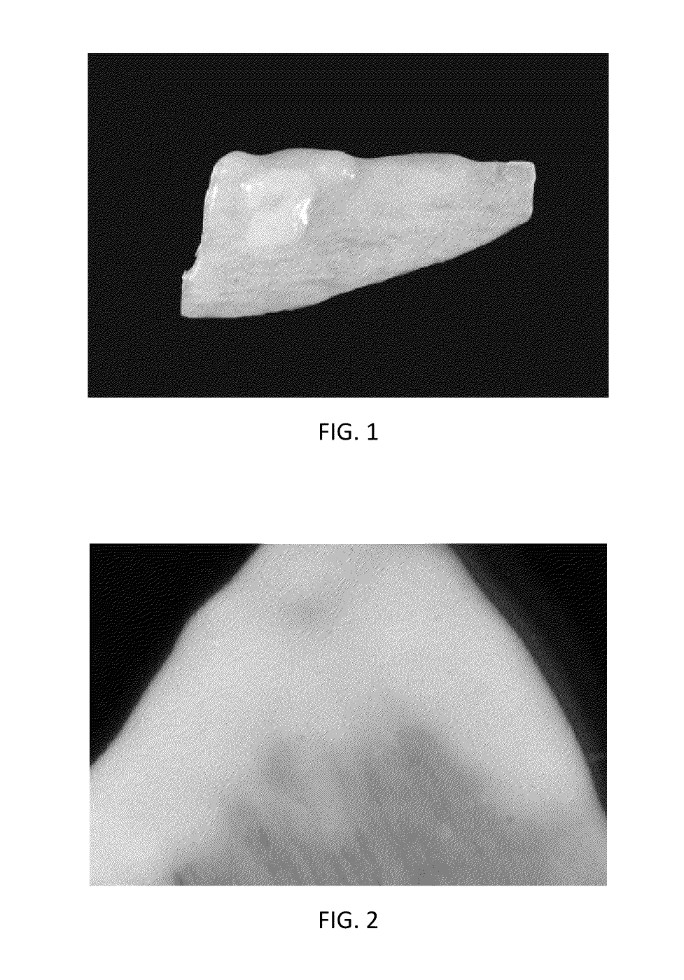

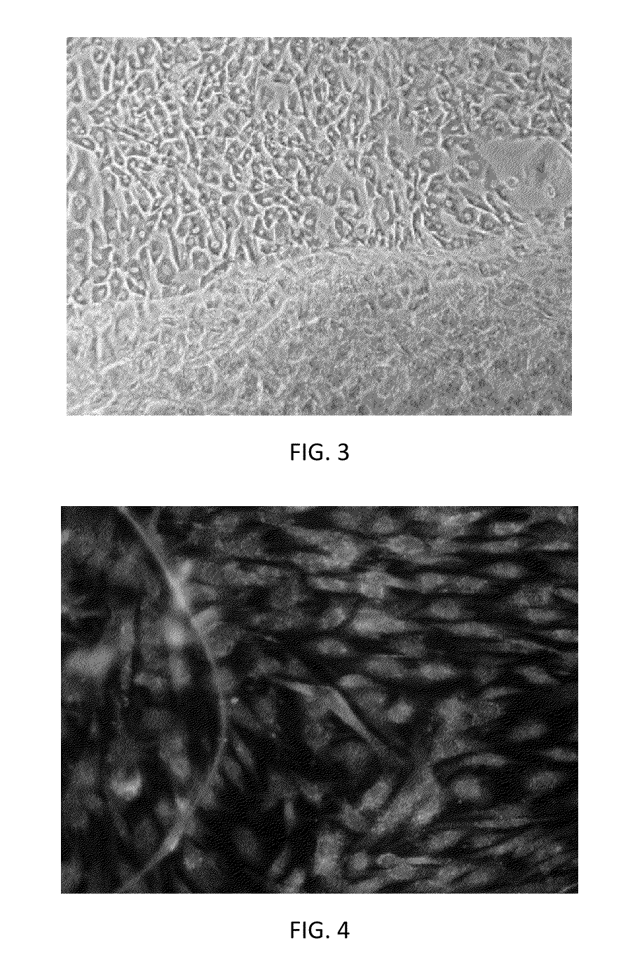

The invention in the present embodiments by-passes the problem with chondrocyte dedifferentiation because it provides for adequate numbers of chondrocytes forming new cartilage on biological substrates and for clinical transplantation without need for chondrocyte expansion. As is shown in FIGS. 1-5. The preparation of chondrocyte cultures is carried out as follows: fresh cartilage slices are prepared with sharp blades from articular cartilage of adult, juvenile, infantile or neonatal articular cartilage. The cartilage is washed in balanced salt solution and placed on Teflon or other suitable boards for further sectioning. Cartilage fragments suitable for placement into cell culture vessels are obtained by cylindrical tubes made into geometric arrays or by cubes produced by blade grids or wire grids. Cartilage plates with cylindrical perforations can be cultured intact and transplanted as such into articular defects. Square or rectangular cartilage pieces can be cultured in a like fashion. The diameter of tubes for obtaining cartilage samples is from 1 to 5 mm. The rectangular pieces likewise measure from 1×1 to 5×5 mm. Larger pieces can be also employed.

For culturing explants, a number of commercially available cell culture media may be employed. These are but not limited to DHEM, NCTC-19, RPMT-1640, CMRL 1415, Eagle's baser media prepared with Hanks' or Earle's saline, human chondrocyte growth medium (411-500), promo cell chondrocyte growth medium etc. Chondrocyte growth media usually contain cytokines, GF-beta and other specific supplements. The cell culture can be supplemented with fetal bovine serum, calf serum or human serum.

“Blendzymes” are another group of substances used in chondrocyte propagation. These too can be used in connection with present invention.

“Blendzyme®” preparations were developed by Roche Diagnostics Corporation (Indianapolis, Ind.) by bacterial fermentation to address the demand for proteases with characterized enzymatic activity and purity. For regulatory purposes, these enzymes are ideal for the manufacturing of tissue engineered substances. Blendzyme® 2 contains a combination of collagenase and neutral protease suitable for harvesting primary chondrocytes from articular cartilage or culture vessels. Reduced of the same enzyme are used when chondrocytes need to be released from a culture substrate.

Blendzymes are combinations of purified collagenases I and II and a neutral protease. The collagenases are purified from the fermentation of Clostridium histolyticum. Currently four formulations are available. Blendzyme® 1 contains the neutral protease dispase, which is purified from Bacillus polymyxa fermentation. Blendzyme® 2, 3 and 4 contain the neutral protease thermolysin, purified from Bacillus polymyxa fermentation, as disclosed in U.S. Pat. Nos. 5,753,485 and 5,830,741.

Cell organization, as applied to chondrocytes and formation of phenotype retaining cartilage is a scientific endeavor actively pursued in regeneration medicine. Studies of cell organization are broadly divided into two groups. One is the study and production of two dimensional cell sheets. The other one is the production and investigation of three dimensional structures such as organized cartilage composed of chondrocytes and intercellular matrix, arranged in multiple layers. Both types of chondrocytic organization are the subjects of present invention. Chondrocytes grown and multiplied on the surfaces of culture vessels are subject to harvesting by mechanical (rubber policemen) or chemical means. These can be harvested either with or without explants from which the cultures originated. For convenience of harvesting and transfer, the explants can be placed on glass coverslips, plastic coverslips, perforated cellophane and biodegradable membranes or plates made of collagen, dermis, facia lata, pericardium or synthetic materials such as polyvinyl pyrrolidone, cellulose and similar substances. Chondrocytes grown on these substances can be segregated by exposure to substances such as Versene®, made into cell pellets and thus prepared for implantation. The advantage of the method is the membranes, plates or other structures can be easily handled by removing them from the culture vessel and treating them in open vessels such as large Petri dishes.

In another embodiment, cartilage explants are placed on thin cancellous bone plates partially immersed into culture medium with fluid covering only the surface. The cells from the explants cover the surface of the bone plate and penetrate into trabecular spaces as shown in FIG. 6. If flexibility of the cancellous bone plate is desired, the bone is decalcified in hydrochloric acid (0.5N). The flexible cancellous bone plate with cartilage cells and newly formed cartilage covering it can be contoured to the defect into which it is placed. Cartilage explants can be also grown on thin cortical bone plates undecalcified or decalcified.

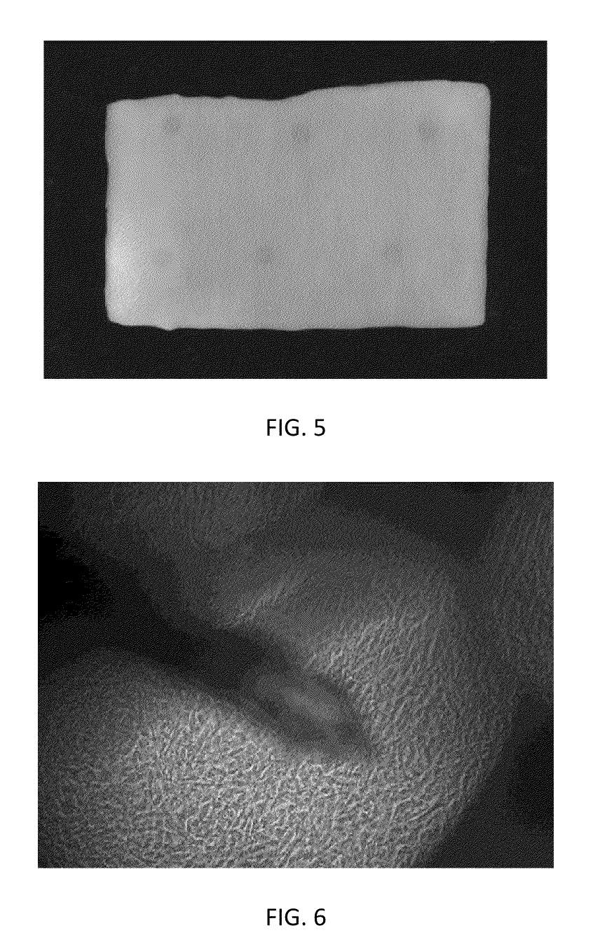

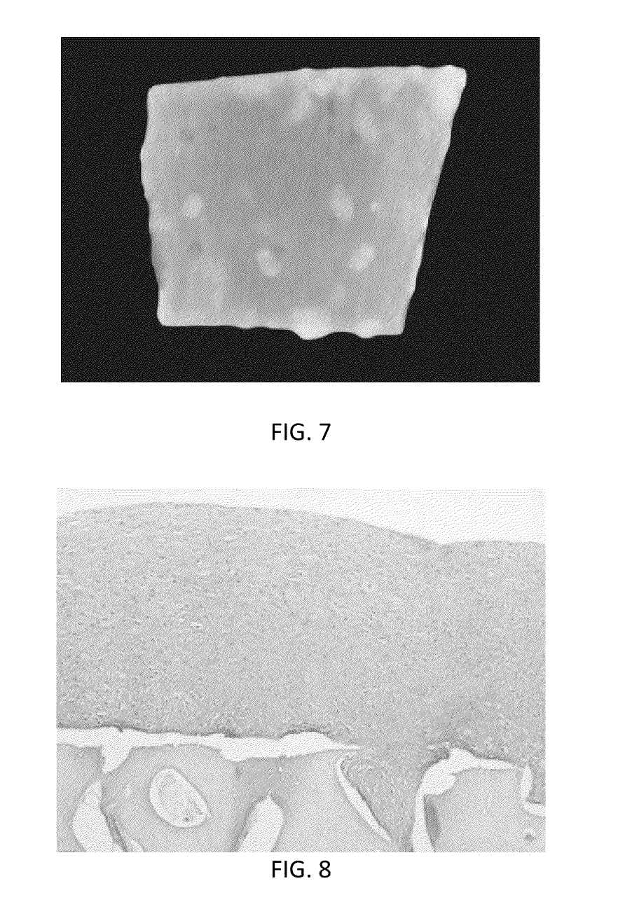

Since cortical bone plates have no natural openings as do cancellous plates perforation can be made in them to allow for the ingrowth of cartilage. Chondrocytes from explants grow on the surface of perforated bone plates and obliterate the artificial perforations as shown in FIGS. 5 and 7. Chondrocytes emanating from cartilage explants form fully structured hyaline cartilage on the bone plates, as shown in FIG. 8.

In another embodiment, cartilage explants can be placed on the surface of reconstituted Gelfoam®, Surgicel®, covered with thrombin or without thrombin. These hemostatic substances are commonly used in surgery. When left in the body they are readily absorbed without undue reaction. To this end Gelfoam or Surgicel with chondrocytes growing on their surfaces can be conveniently placed into articular cartilage defects with or without residual explant tissue.

In another embodiment, explants can be placed on sheets of decellularized dermis and transferred into articular cartilage defects with the newly grown layer of chondrocytes. Other biological degradable membranes which can be used in a similar manner are pericardium and fascia's including fascia lata, and dura mater.

In another embodiment cartilage cells from explants can be grown on the surfaces of synthetic membranes such as collodion, hydroxyethyl starch, polyvinylpyrrolidone, perforated cellophane and similar preparations.

In another embodiment, chondrocytes and the cartilage explants growing on any of the surfaces can be harvested mechanically or enzymatically, frozen, then either freeze-dried or subjected to hypothermic dehydration and micronized. Preparations can be used for implantation into articular defects to stimulate cartilage regeneration from the host.

What is claimed is:

1. A method for production of large numbers of cartilage cells comprises the step of:
preparing viable cartilage tissue by propagation of chondrocytes from cartilage explants, human or animal, with the retention of phenotypes of the cartilage cells.

2. The method according to claim 1 in which cells are chondrocytes retaining morphologic attributes of the same or chondrocyte progenitor cells.

3. The method according to claim 1 in which cultured cells are organized into mature hyaline cartilage on the surface of biologic structures such as cancellous or cortical bone lamina.

4. The method according to claim 1 for culturing chondrocytes to produce three dimensional cellular structure consisting of cartilage cells and cell produced extracellular cartilage matrix.

5. The method according to claim 1 whereby biologic material comprising hyaline cartilage tissue is propagated on the surface of thin cancellous bone which is undecalcified, and wherein the said cancellous bone lamina is from 0.5 to 2.0 mm in thickness.

6. The method according to claim 1 whereby cancellous bone lamina, 0.5 to 2.0 mm thick is decalcified to render it flexible.

7. The method according to claim 1 whereby cancellous bone lamina is either demineralized or undemineralized and is thicker than 2.0 mm.

8. The method according to claim 5 whereby newly proliferating cartilage can be transplanted into a chondral defect together with the underlying substrate without expanding the cells.

9. The method according to claim 6 whereby newly proliferating cartilage can be transplanted into a chondral defect together with the underlying substrate without expanding the cells.

10. The method according to claim 7 whereby newly proliferating cartilage can be transplanted into a chondral defect together with the underlying substrate without expanding the cells.

11. The method according to claim 1 whereby a substrate on which chondrocytes proliferate from explants is a lamina of undecalcified cortical bone 0.5 to 2.0 mm thick with multiple, geometrically placed perforations.

12. The method according to claim 1 whereby the lamina on which explants are placed is decalcified cortical bone plate 0.5 to 2.0 mm thick with multiple, geometrically placed perforations.

13. The method according to claim 1 whereby all osseous substrates on which chondrocytes have proliferated from cartilage explants are cryopreserved for further transplantation.

14. The method according to claim 1 whereby cartilage explants are cultivated on a cellular dermis, pericardium, dura mater and fascia with or without perforations.

15. The method according to claim 1 whereby chondrocytes proliferated on soft membranes with or without perforations are cryopreserved for future transplantation.

16. The method according to claim 1 whereby cartilage explants are placed on Gelfoam®, Surgicel®, Gelatin USP, and or similar materials which can be optionally supplemented with thrombin or ligrin.

17. The method according to claim 1 whereby cartilage explants are grown on membranes of collodion, polyvinylpyrrolidone, hydroxyethyl starch or an equivalent thereof.

18. The method according to claim 1 whereby cartilage cells together with or without cartilage explants are harvested mechanically, enzymatically or physically from the substrates on which they are grown, frozen and either freeze-dried or dehydrated under hypothermia and micronized for implantation into articular defects.

19. The method according to claim 18 wherein cells and explants can be frozen with substrates and then later scraped with a blade for further processing.
